(19) United States
(12) Patent Application Publication
(10) Pub. No.: US 2022/0162090 A1
(43) Pub. Date: May 26, 2022

(54) RAPID INLINE DIFFERENTIAL WATER ANALYZER

(71) Applicant: United States of America as Represented by The Secretary of The Army, Alexandria, VA (US)

(72) Inventors: Tung N. Ly, Lorton, VA (US); Donald M. Cropek, Champaign, IL (US); Michael J. Anderson, Detroit, MI (US)

(21) Appl. No.: 17/102,007

(22) Filed: Nov. 23, 2020

(51) Int. Cl. C02F 1/00; C02F 1/44; G06Q 10/06; G06Q 50/26; G05B 19/042; G01N 33/18

(52) U.S. Cl. CPC C02F 1/008; G05B 2219/25257; G06Q 10/06395; G06Q 50/26; G05B 19/0423; G01N 33/18; C02F 2209/006; C02F 2209/008; C02F 2209/02; C02F 2209/22; C02F 2209/06; C02F 2209/05; C02F 2209/04; C02F 2209/11; G05B 2219/25252; C02F 1/441

(57) ABSTRACT

In one embodiment, a differential water data analysis system includes a data storage, a user interface, and a processor coupled to the data storage and programmed with software to: receive inflow water measurement data from a water inflow line entering a water treatment system and outflow water measurement data from a water outflow line exiting the water treatment system, the inflow water measurement data and outflow water measurement data being obtained by measuring the water in the water inflow line and the water outflow line at about the same time and being received in real time; compare the inflow water measurement data and the outflow water measurement data; compute a performance of the water treatment system based on the comparison using a computation scheme; determine whether the performance is within specification of the water treatment system to produce a determination output; and display the determination output in the user interface.

### STATEMENT OF GOVERNMENT INTEREST

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees.

### BACKGROUND

### Field of the Invention

The present invention relates to apparatus and methods of evaluating the performance of water filtration or treatment systems and, more particularly, to a differential water analyzer that can be readily connected to and disconnected from a variety of water filtration systems.

### Description of the Related Art

This section introduces aspects that may help facilitate a better understanding of the invention. Accordingly, the statements of this section are to be read in this light and are not to be understood as admissions about what is prior art or what is not prior art.

Filtration is a separation process that involves passing a solid-liquid mixture through a porous material (filter) which retains the solids and allows the liquid (filtrate) to pass therethrough. Reverse osmosis (RO) is a water purification or treatment process that uses pressure to force water molecules through a partially permeable membrane to remove contaminants such as ions, unwanted molecules, and larger particles. Filters and membranes need to be periodically replaced to ensure performance of a water filtration system. Replacing them too early increases cost while replacing them too late decreases performance. In addition, components of the filtration system may deteriorate or fail. Evaluating or monitoring the performance of the water filtration system is desirable to ensure performance and control replacement and repair cost. In this disclosure, water filtration and water treatment are used interchangeably and encompass all methods of processing water to change its qualities or characteristics.

### SUMMARY

The present invention was developed to address the desire for a versatile and convenient system for quickly and accurately evaluating the performance of water filtration systems. Research and development have led to a novel system that may be a single, inline portable unit which can be connected to and disconnected from a variety of water filtration systems and quickly evaluate their performances without halting or otherwise interfering with the operation of the filtration systems.

Embodiments of the present invention provide an inline differential water data analysis system and method for water filtration system diagnostics, quality control, and/or performance monitoring. The system is configured as a proper SWaP-C (Size, Weight and Power plus Cost) self-contained software-driven system. The system performs differential measurements of water quality parameters and data analyses of the water entering and the water exiting the filtration system. The data measured, analyses performed, and results generated are shown in real time on a local display. Compared to the present system which may be a single portable unit, prior systems require multiple, separate system assemblies to do differential measurements, and are larger, more costly, and less flexible. The inline differential water data analysis system can be removably connected to any of a variety of water filtration systems ranging from factory systems to field systems.

The present invention advances the science of performance evaluation of water treatment systems. Key to the success of this apparatus is, among others, the ability to conduct rapid inline differential water data analysis of the water treatment system in real time by obtaining differential water measurement data from sensors inserted into the water filtration process streams upstream and downstream of the filtration, comparing and analyzing the data, evaluating the performance of the water treatment system, and displaying and/or monitoring the result, all in real time.

According to an aspect the present invention, a differential water data analysis system comprising a data storage, a user interface, and a processor coupled to the data storage and programmed with software to: receive inflow water measurement data from a water inflow line entering a water treatment system and outflow water measurement data from a water outflow line exiting the water treatment system, the inflow water measurement data and outflow water measurement data being obtained by measuring the water in the water inflow line and the water outflow line at about the same time and being received in real time; compare the inflow water measurement data and the outflow water measurement data to obtain a comparison; compute a performance of the water treatment system based on the comparison using a computation scheme; determine whether the performance of the water treatment system is within or outside a specification of the water treatment system to produce a determination output; and display the determination output in the user interface. The processor may perform the tasks in real time with measuring the water in the water inflow line and the water outflow line by sensors.

In some embodiments, a water inflow data line forms a releasable connection with a water inflow sensor disposed in the water inflow line to measure the inflow water measurement data; and a water outflow data line forms a releasable connection with a water outflow sensor disposed in the water outflow line to measure the outflow water measurement data. The water inflow sensor and water outflow sensor are inline sensors to perform inline measurement of the water in the water inflow line and the water outflow line, respectively, the inflow water measurement data and outflow water measurement data being inline measurement data obtained without removing a water sample out of the water treatment system or the water inflow line or the water outflow line for measurement.

In specific embodiments, the inflow water measurement data and the outflow water measurement data each comprise data relating to one or more water quality parameters and the specification of the water treatment system comprises a mathematical expression which includes the one or more water quality parameters.

In some embodiments, the processor is programmed, when the performance of the water treatment system is outside the specification of the water treatment system, to produce an alarm indicating existence of performance inadequacy of the water treatment system, and/or perform at least one of operation logic modification to modify operation logic of the inline differential water data analysis system, data computation modification to modify the computation scheme of the inline differential water data analysis system, or data logging modification to modify data logging of the inflow water measurement data and the outflow water measurement data by the inline differential water data analysis system.

In specific embodiments, a data carrier is configured to provide data communication with a remote monitoring device. The processor is programmed to receive (e.g., in real time) at least one of (i) user input including instruction for the inline differential water data analysis system or (ii) software update of the software for the processor, from at least one of the user interface or the data carrier.

In accordance with another aspect of the invention, a portable differential water data analysis system comprises: a data storage; a user interface; a water inflow data line forming releasable connection with a water inflow sensor disposed in a water inflow line entering a water treatment system to collect inflow water measurement data of the water treatment system; a water outflow data line forming releasable connection with a water outflow sensor disposed in a water outflow line exiting the water treatment system to collect outflow water measurement data of the water treatment system, the inflow water measurement data and outflow water measurement data being obtained by measuring the water in the water inflow line and the water outflow line at about the same time and being collected in real time; and a processor coupled to the data storage and programmed with software to compare the inflow water measurement data and the outflow water measurement data to obtain a comparison; compute a performance of the water treatment system based on the comparison using a computation scheme; determine whether the performance of the water treatment system is within or outside a specification of the water treatment system to produce a determination output; and display the determination output in the user interface. The processor may perform the tasks in real time with measuring the water in the water inflow line and the water outflow line by sensors.

Yet another aspect of this invention is directed to a differential water data analysis method for a differential water data analysis which includes a data storage, a user interface, and a processor programmed with software. The method comprises: receiving inflow water measurement data from a water inflow line entering a water treatment system and outflow water measurement data from a water outflow line exiting the water treatment system, the inflow water measurement data and outflow water measurement data being obtained by measuring the water in the water inflow line and the water outflow line at about the same time and being received in real time; comparing the inflow water measurement data and the outflow water measurement data to obtain a comparison; computing a performance of the water treatment system based on the comparison using a computation scheme; determining whether the performance of the water treatment system is within or outside a specification of the water treatment system to produce a determination output; and displaying the determination output in the user interface.

Embodiments of the differential water data analysis system enable rapid differential water data analysis of a water treatment system in real time using a single portable software-driven device, without removing a water sample out of the water treatment system or the water inflow line or the water outflow line for measurement or diverting the water flowing through the water inflow line and the water treatment system and the water outflow line to a side stream for measurement.

Embodiments of the differential water data analysis system can be used for a variety of applications including, for example, analyses of drinking water production, troubleshooting for water logistic engineers or military quarter masters, water testing for long-term storages, military applications involving logistics and mission commands, civil work involving municipalities and environmental monitoring and protection, and humanitarian assistance and disaster relief.

### BRIEF DESCRIPTION OF THE DRAWINGS

Embodiments of the invention will become more fully apparent from the following detailed description, the appended claims, and the accompanying drawings in which like reference numerals identify similar or identical elements.

FIG. 1 schematically illustrates an environment in which an inline differential water data analysis system operates to analyze water entering into and exiting from a water filtration system according to an embodiment of the present invention.

FIG. 2 is a block diagram of the inline differential water data analysis system according to an embodiment of the invention.

FIG. 3 is a flow diagram illustrating a process of operating the inline differential water data analysis system according to an embodiment of the invention.

FIG. 4 is a diagram illustrating differential water data collection by the inline differential water data analysis system according to an embodiment of the invention.

FIG. 5 is a system logic diagram of the inline differential water data analysis system according to an embodiment of the invention.

FIG. 6 depicts an exemplary computer system or device configured for use in the water data analysis system according to an embodiment of the present invention.

### DETAILED DESCRIPTION

Detailed illustrative embodiments of the present invention are disclosed herein. However, specific structural and functional details disclosed herein are merely representative for purposes of describing example embodiments of the present invention. The present invention may be embodied in many alternate forms and should not be construed as limited to only the embodiments set forth herein. Further, the terminology used herein is for the purpose of describing particular embodiments only and is not intended to be limiting of example embodiments of the invention.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Embodiments of the present invention provide a single portable inline differential water data analysis system for differential water data analysis of a water filtration system by obtaining inflow water measurement data from a water inflow line entering the water treatment system and outflow water measurement data from a water outflow line exiting the water treatment system, without removing a water sample out of the water treatment system or the water inflow line or the water outflow line for measurement, comparing the data, computing a performance of the water treatment system, determining whether the performance of the water treatment system is within or outside a specification, and displaying and/or monitoring the output in real time.

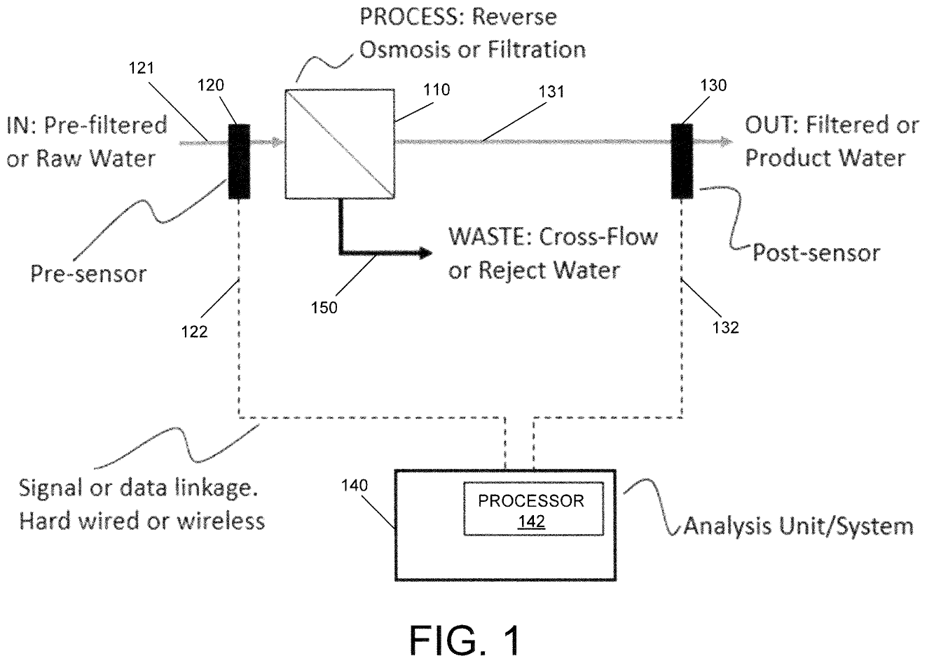

FIG. 1 schematically illustrates an environment in which an inline differential water data analysis operates to analyze water entering into and exiting from a water filtration system according to an embodiment of the present invention.

A water filtration system 110 receives an inflow of pre-filtered or raw water in a water inflow line 121 entering the system 110 and produces an outflow of filtered or product water in a water outflow line 131 exiting the system 110. A pre-sensor 120 is disposed upstream of the filtration system 110 in the inflow line 121 to measure the pre-filtered water properties. A post-sensor 130 is disposed downstream of the filtration system 110 in the outflow line 131 to measure the filtered water properties. An analysis unit or system 140 receives signals or data via signal or data linkages which may be hard-wired or wireless. For instance, it receives data from the pre-sensor 120 via a water inflow data line 122 coupled thereto and from the post-sensor 130 via a water outflow data line 132 coupled thereto. According to an embodiment of the invention, the data analysis system 140 includes a rapid inline differential water analyzer (RIDWA) which has a processor/logic 142 programmed with software to process the input data or signals. The water inflow data line 122 and water outflow data line 132 are any suitable data communication lines for transmitting or receiving data.

The inflow water measurement data and outflow water measurement data are obtained by measuring the water in the water inflow line 121 using the pre-sensor 120 and the water outflow line 131 by the post-sensor 130 at about the same time, for example, within about a minute, or within a few seconds, or simultaneously.

Typically, the pre-sensor 120 and post-sensor 130 are not inherent components of the water filtration system 110 but are external components coupled to the filtration system 110 to provide measurement input to the differential water analyzer. The sensors are inserted into the water filtration process streams. The system can use COTS (Commercial Off The Shelves) sensors that are standardized in the market and use typically 2-wire connections. Alternatively, the actual connector can be adapted through connection adapters instead of the 2-wire format. On the other hand, if the sensors are already part of the filtration system, the differential water analyzer can utilize those sensors or sensor pair to obtain water measurement inputs, provided they are non-proprietary sensors.

Typically, insertion of the sensors into the water filtration process streams is done before the filtration system 110 is fluidized and would remain in place for continuous monitoring or until the differential water data analysis and evaluation is completed. The insertion can be done by a variety of ways. In one example, a compression fitting such as an O-ring is placed around a circular cross-sectioned sensor and a compression collar is used to hold the probe in place. In another example, one or more sensors are integrated into a plumbing fitting as art of their design and are then integrated into the flow path plumbing. In yet another example, one or more sensors are configured as a sanitary fitting to be integrated into a tee which is pre-plumbed in for the filtration system water stream.

In one example, the water inflow data line 122 forms a releasable communication or connection with the pre-sensor 120 which is inserted in or otherwise connected to the water inflow line 121 and the water outflow data line 132 forms a releasable communication or connection with the post-sensor 130 which is inserted in or otherwise connected to the water outflow line 131. These sensors remain in place and can be used for continuous monitoring. This is an example of inline sensing and measurement using inline sensors.

In another embodiment which is non-inline sensing and measurement, the pre-sensor 120 and post-sensor 130 are detachably coupled to the water filtration system 110. To connect and disconnect the probes or sensors without stopping water flow in the filtration system 110 or otherwise interfering with operation of the filtration system 110, one approach is to create a bypass loop which is a section of water flow that diverts from the main water stream, runs parallel to the main water stream, and is then returned to main stream. The pre-sensor 120 is connected to and disconnected from an upstream bypass loop upstream of filtration and the post-sensor 130 is connected to and disconnected from a downstream bypass loop downstream of filtration. The entrance to the bypass loop may be controlled by valve(s). The bypass loop is called a side stream. Alternatively, the water sample used for sensor measurement is discarded using a sampling port instead of a side stream flowing through a bypass loop. The pre-sensor 120 is connected to and disconnected from an upstream sampling port upstream of filtration and the post-sensor 130 is connected to and disconnected from a downstream sampling port downstream of filtration.

FIG. 1 shows a waste stream 150 exiting the filtration system 110. Not all filtration systems produce a waste stream. For example, a filter may not have a waste stream, but rather remove and concentrate the material on the filter. The wastewater exiting the filtration system 110 in the waste stream 150 has the suspended solids removed and can be measured via turbidity. Turbidity is a common index for filtration efficiency determination; however, this index is weak for particles with diameter of 1-5 μm including pathogen microorganisms. The EPN (escaped particle number) and turbidity can be used to monitor filtration efficiency.

The filtration system 110 uses any available mechanism or process of removing a portion of material(s) in a water stream. Performance of the removal process can be measured using any available sensing device or method, including the use of the pre-sensor 120 and the post-sensor 130. The pre-sensor 120 and the post-sensor 130 each may include a set or combination of multiple sensors.

In this embodiment, sensors are used in sets to obtain differential data. Some sensors can be a combination of sensors, such as temperature correcting pH electrodes. Raw sensor data can be sent via corded connection or wirelessly. The mode of data transmission does not matter. Differential measurements of conductivity, specifically, can help provide information about the equipment.

Examples of what the sensor senses include, but are not limited to, conductivity, resistance, impedance, pH, turbidity, dissolved oxygen (DO), temperature, oxidation reduction potential (ORP), pressure, volumetric flow, light absorbance/transmittance and/or color. Essentially any suitable device can be used to measure a property of water, be it electrical, chemical, or bulk property (flow, temperature, pressure, etc.). Specific examples include water flow cell /module for electrodes/probes as well as conductivity probes and temperature probes.

The measurement of differential water quality parameters is not commonly done because of how regulations are written. The feed water is often not measured except for finding the “best” starting water and product water is regulated with fixed amounts/values to not exceed. Therefore, if measurements are automated, they are done to ensure that the water coming out falls under these limits (e.g., Environmental Protection Agency drinking guidelines, World Health Organization guidelines, or Military TB MED 577 guidelines).

In this embodiment, the inline differential water data analysis system 140 includes a rapid inline differential water analyzer (RIDWA) which performs inline measurement, using inline sensors or probes that are located in the water flow path of the filtration system 110 and are part of a continuous sequence of operations of the filtration system 110. The pre-sensor 120 is located in the inflow portion and the post-sensor 130 is located in the outflow portion of the water flow path through the filtration system 110. The inline measurement does not require removing a water sample (or any fluid sample) from the filtration system 110 for measurement. No special flow changes are required to redirect a fluid sample for measurement. Nor is the filtration system 110 required to be stopped for measurement.

In specific embodiments, the water inflow data line 122 forms a releasable communication or connection with the water inflow sensor 120 disposed in the water inflow line 121 to measure the inflow water measurement data and the water outflow data line 132 forms a releasable communication or connection with the water outflow sensor 130 disposed in the water outflow line 131 to measure the outflow water measurement data. The inflow water measurement data and the outflow water measurement data each comprise data relating to one or more water quality parameters.

In contrast, non-inline measurement would require manually taking a water sample out of the filtration system 110 or the water inflow line 121 or the water outflow line 131 to different containers for measurement or mechanically diverting (e.g., by pumping) the main flow of water through the water inflow line 121 and the water filtration system 110 and the water outflow line 131 to a small side-stream for measurement. These non-inline methods would be less accurate and less timely because the parameters (e.g., temperature, flow pressure, ions exchanges/reactions, molecular concentration, etc.) would change outside of the dynamic environments in which the water flows originally through the filtration system. Moreover, many if not most of the measurements are inter-correlated and dependent on each other (i.e., conductivity vs. temperature vs. pH, chlorine vs. flow vs. temperature, etc.). As such, the measurement inaccuracies may be compounded.

Water quality parameters include chemical, physical, and biological properties and can be tested or monitored based on the desired water parameters of concern. Parameters that are frequently sampled or monitored for water quality include temperature, DO, pH, conductivity, ORP, and turbidity. However, water monitoring may also include measuring total algae, Ion Selective Electrodes (ISEs) (e.g., ammonia, nitrate, chloride), or laboratory parameters such as Biological Oxygen Demand (BOD), titration, or Total Organic Carbon (TOC).

Monitoring the change in water quality parameters is not very effective on only filtration-based purification systems. This is because filters alone can remove only suspended solids in water, such as bacterial, algae, dirt, plant matter, and other larger items. Even the most extreme of the filters, the ultrafilter (UF), do not remove salts and other small molecules such as an RO (reverse osmosis) membrane. Ultrafilters are used in dialysis to remove salts from blood by allowing the salts to diffuse through the ultrafilter into clean solutions following the osmotic potential. Reverse osmosis membranes force water against the osmotic potential (i.e., by restricting salts, ions, and individual atoms) to produce water. It is primarily these small ions/atoms that change the chemistry of the water. During the RO process, water is usually made more acidic, conductivity is reduced, and reduction in Oxidation Reduction Potential (ORP) is achieved.

ORP is very important in drinking water. ORP is typically measured to determine the oxidizing or reducing potential of a water sample, i.e., reducing and oxidizing agents (redox). It indicates possible contamination, especially by industrial wastewater. ORP can be valuable if the user knows that one component of the sample is primarily responsible for the observed value. For example, excess chlorine in wastewater effluent will result in a large positive value and the presence of hydrogen sulfide will result in a large negative value. ORP affects other water quality measurements.

ORP is determined by measuring the potential of a chemically inert (e.g., platinum) electrode which is immersed in the solution. The sensing electrode potential is read relative to the reference electrode of the pH probe and the value is presented in millivolts (mV). The determination of ORP is generally significant in water which contains a relatively high concentration of a redox-active species, e.g., the salts of many metals (Fe²⁺, Fe³⁺) and strong oxidizing (chlorine) and reducing (sulfite ion) agents. Thus, ORP can sometimes be utilized to track the metallic pollution in groundwater or surface water or to determine the chlorine content of wastewater effluent. However, ORP is a nonspecific measurement, i.e., the measured potential is reflective of a combination of the effects of all the dissolved species in the medium. Because of this factor, the measurement of ORP in relatively clean environmental water (ground, surface, estuarine, and marine) has only limited value unless a predominant redox-active species is known to be present.

The value of redox in determining the content of environmental water is greatly enhanced if the user has some knowledge or history of the site. ORP data can typically become more useful if used as an indicator over time and/or with other common parameters to help develop a complete picture of the water quality being tested.

Monitoring of ORP could be done somewhat effectively with filters only, as many organic molecules can be removed. The effects of filtration on total dissolved solids (TDS) which is measured in parts per million (ppm) is not very effective. Some larger molecules do contribute to conductivity, but in water purification this is a very small contribution of the TDS compared to mineral salts and compounds. The effect on pH is similar to TDS with filters only.

Electrical conductivity is an indicator of water quality. Conductivity data can determine concentration of solutions, detect contaminants, and determine the purity of water. YSI conductivity sensors measure conductivity by Alternating Current (AC) voltage applied to nickel electrodes. These electrodes are placed in a water sample (or other liquid), where the current flows through the electrodes and the sample. Current level has a direct relationship with the conductivity of the solution.

Conductivity is the ability of a material to conduct electrical current. The principle by which instruments measure conductivity is simple. Plates or wires are placed in the sample, a potential is applied across them (normally a sine wave voltage), and the current is measured. Conductivity, the inverse of resistivity, is determined from the voltage and current values according to Ohm's law. Since the charge on ions in solution facilitates the conductance of electrical current, the conductivity of a solution is proportional to its ion concentration. In some situations, however, conductivity may not correlate directly to concentration. Ionic concentrations can alter the linear relationship between conductivity and concentration in some highly concentrated solutions. The basic unit of conductivity is the siemens (S), sometimes referred to as ohm. Since cell geometry affects conductivity values, standardized measurements are expressed in specific conductivity units (S/m or μS/cm) to compensate for variations in electrode dimensions.

Conductivity meters can use different types of sensors such as non-wiped conductivity field sensor. It involves measuring electrical current passing through water. It can show presence of inorganic dissolved solids such as chloride, nitrate, sulfate, and phosphate negative ions or sodium, magnesium, calcium, iron, and aluminum positive ions. Conductivity measurements indicate the physical quality of water and removal of pollution by the water filtrations system. Conductivity measurements are temperature dependent and, along with temperature, also allow for water salinity values to be calculated through algorithms.

Visual, photometric, and potentiometric methods can be used to measure the hydrogen ion activity of a solution. Visual and photometric methods rely on color changes of specific organic pigments in order to determine pH. Visual methods are completed with visual indicators such as pH test strips or color camera, while photometric determination involves shining a light through the sample and measuring the absorbance. The application of visual or photometric determination of pH is limited. Measurements will be unreliable if the solution to be measured is cloudy, has an inherent color, or is stagnant. Some measurement solutions also contain chemical bonds which destroy the color indicators through oxidation or reduction and produce incorrect results. The alkalinity or acidity of water is an indication of pollution and toxicity of water. The pH value affects other water quality measurements.

Potentiometric methods determine pH by using the electrical potential of pH-sensitive electrodes as a measurement signal, which is then displayed by a pH meter. The disadvantages of visual and photometric methods are not present with potentiometric methods, as potentiometric sensors are very sensitive, selective, and can be used in almost any application.

The glass pH sensor is an example of an ion selective electrode (ISE). This system consists of the ISE reacting on a special ion type, in this case the hydrogen ion, and a reference electrode that are jointly immersed in the sample to be measured. A pH electrode is technically a hydrogen ISE. The only major difference between a pH electrode and a nitrate ISE is the membrane used. The hydrogen ISE provides an electrochemical potential (e.g., signal) that is influenced by the hydrogen ion activity of the solution. The reference electrode, however, maintains an electrochemical potential that does not depend on the composition of the sample. The difference between these potentials, the voltage (mV) displayed on a pH meter, determines the pH value based on the Nernst equation.

Temperature affects biological and chemical processes in water. Temperature has an effect on other water quality measurements.

Dissolved oxygen (DO) affects biological and chemical processes in water. It is an indication of pollution in water.

Turbidity is a measurement of water clarity and results from small materials that are suspended in water. Turbidity is an indication of pollution in water and affects biological and chemical processes in water and is measured in Nephelometric Turbidity Units (NTU).

The sensors are capable of water conductivity, salinity, dissolved ions/solids, and temperature measurements in single or differential modes. They provide monitoring, performance validation, and diagnostics of COTS systems for water filtration, purification, and desalination. Sensor hardware may have the following characteristics: low-cost, low-power, network-enabled, reprogrammable, COTS probes compatible, with multiple data storage options and multiple input/output (I/O) options. Sensor software may have the following characteristics: data standards, real-time monitoring, modular-extensible, differential analyses, support geospatial applications, and software-driven calibrations.

The inline differential water data analysis system 140 provides real-time differential remote monitoring and data analyses. It is characterized by data open standards and adaptability to using a variety of COTS low-cost sensor probes. The operational environment may involve surface or storage water testing as well as laboratory or industrial processes.

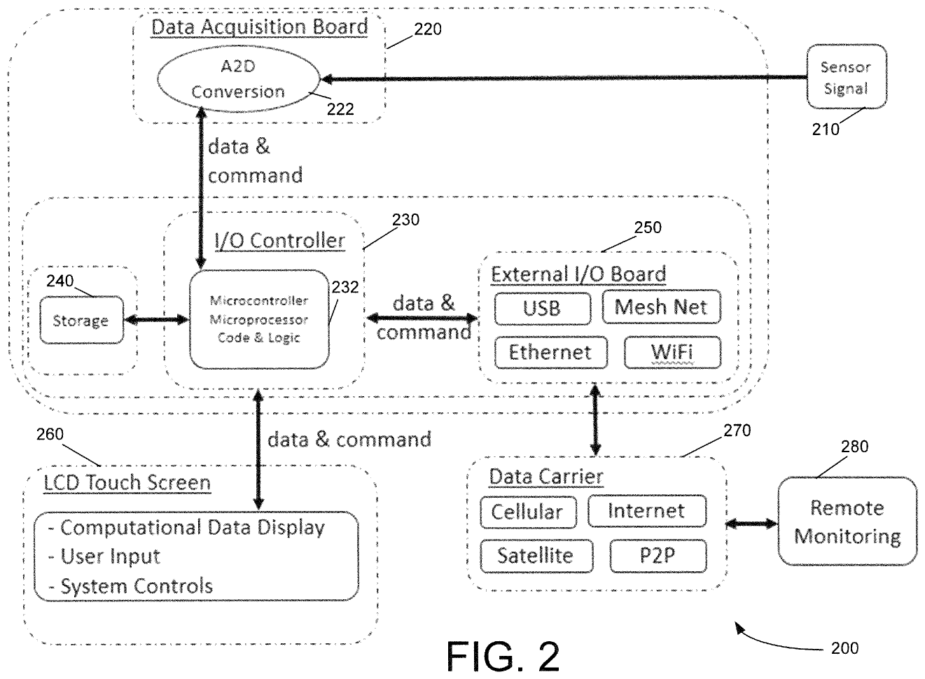

FIG. 2 is a block diagram 200 of the inline differential water data analysis system 140 from FIG. 1 according to an embodiment of the invention. A sensor signal 210 is received by a data acquisition board 220 which includes an A2D (analog-to-digital) conversion device 222 to convert the analog sensor measurement signal 210 from the pre-sensor 120 and post-sensor 130 into digital form for storage and/or processing.

An input/output (I/O) controller 230 is in communication with non-transitory data storage 240 for data flow or transfer and in communication with the data acquisition board 220, external I/O board 250, and user interface 260 for data and command flow or transfer. The storage 240 may include, without limitation, local and/or network accessible storage, a disk drive, a drive array, an optical storage device, a solid-state storage device, such as a random access memory and/or a read-only memory, which may be programmable or flash-updateable. Such storage devices may be configured to implement any appropriate data stores, including various file systems, database structures, or the like.

The I/O controller 230 includes microcontroller or microprocessor code and logic 232. The hardware elements may be electrically coupled via, for example, a bus. The hardware elements may include a processing unit with one or more processors. The logic 232 may be a special-purpose processor programmed specially to process the differential sensor data, by comparison, computation, and/or the like, and produce evaluation results of the performance of the water filtration system 110.

The user interface 260 includes user input and output devices. In the embodiment shown, the user interface 260 is an LCD touch screen to provide, in real time, computational data display, user input, system controls, and the like.

The external I/O board 250 is in communication with data carrier 270 for data flow. The external I/O board may provide interface in the form of Universal Serial Bus (USB), mesh net, Ethernet, WiFi, and the like. The data carrier 270 provides data communication with a remote monitoring device 280 for data flow. The data carrier 270 may provide interface in the form of cellular, Internet, satellite, peer-to-peer (P2P), and the like. The remote monitoring is optional; the system may be self-contained instead.

In addition to or instead of the communication components described above, the system may include a communications subsystem in the form of a modem, a network card (wireless and/or wired), an infrared communication device, a wireless communication device and/or a chipset such as a Bluetooth device, 802.11 device, Wi-Fi device, WiMAX device, cellular communication facilities such as GSM (Global System for Mobile Communications), W-CDMA (Wideband Code Division Multiple Access), LTE (Long Term Evolution), and the like.

In the embodiment shown in FIG. 2, all the components may be COTS that are small in size and low in cost, which allows the inline differential water data analysis 140 to be a portable device. For example, they include a microprocessor board (Arduino at $35 or Raspberry Pi at $40), a generic data acquisition device (Analog-Digital) that can be generic and upgradable to a higher performance chipset, cheap and reliable Secure Digital (SD) cards for storage, low-cost external I/O peripherals that can be upgraded to more advanced technologies, and low-cost touch screen and/or user interfaces and features that can be upgraded via software.

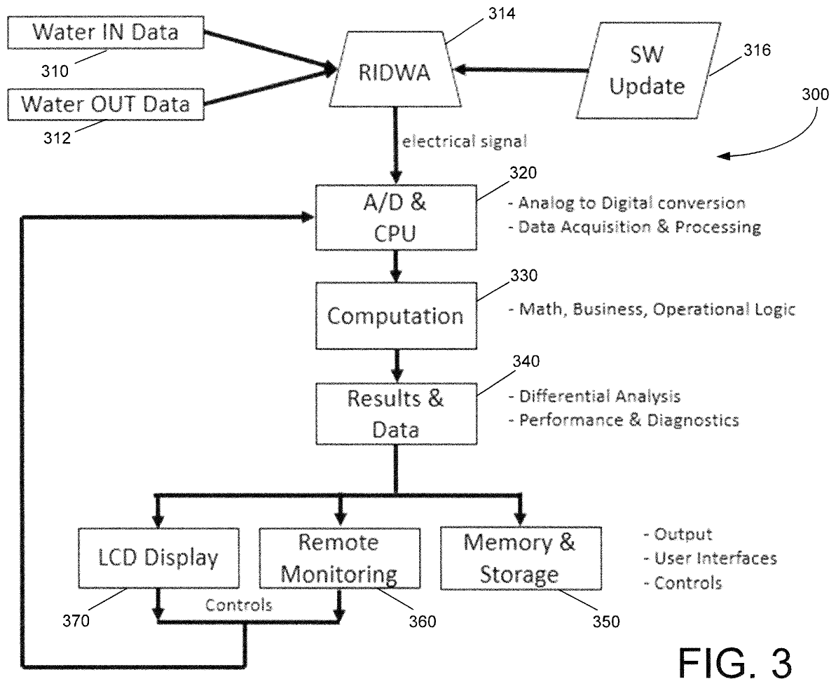

FIG. 3 is a flow diagram 300 illustrating a process of operating the inline differential water data analysis system according to an embodiment of the invention. The inflow water measurement data 310 from the pre-sensor 120 and outflow water measurement data 312 from the post-sensor 130 are received by the analysis system 140 such as RIDWA 314, which also receives any software (SW) update 316. The inflow water measurement data 310 and the outflow water measurement data 312 may be obtained by measuring the water in the water inflow line 121 and the water outflow line 131 simultaneously or at least at about the same time.

The RIDWA 314 is programmed to process the input data and evaluate the performance of the filtration system 110. It includes analog-to-digital conversion and data acquisition and processing by analog/digital (A/D) & central processing unit (CPU) 320 of electrical signals, as performed by the data acquisition board 220 and I/O controller 230. The ND & CPU processing 320 performs analog-to-digital signals conversion from raw analog (voltage, current, resistance, etc.) waveforms to binary data. This is the main data acquisition process, from the electrodes/probes to the CPU. The microprocessor 232 is specially programmed to perform computation 330 on the input data using mathematics, business algorithm, operational logic, or the like. It compares the inflow water measurement data and the outflow water measurement data to obtain a comparison and computes a performance of the water treatment system based on the comparison using a computation scheme.

The inline differential water data analysis and the filtration system performance and diagnostics conducted generates results and data. Results and data processing 340 uses codes and logic to provide output of differential and single data analyses, via user interfaces and data input/output logic. The results include a determination output from determining whether the performance of the water treatment system is within or outside a specification of the water treatment system. The specification of the water treatment system may comprise a mathematical expression which includes the one or more water quality parameters. The output is stored in memory and storage 350 (e.g., raw measurements, results, alarms, or the like stored in storage 240), is provided to remote monitoring 360 (e.g., via data carrier 270 to remote monitoring device 280), and/or is displayed on a display user interface 370 (e.g., via liquid crystal display (LCD) touch screen user interface 260) to show results and alarms and to accept user input such as commands and controls, in real time. For remote monitoring, data streams similar to those directed to the LCD display will go to the peripheral I/O connections such as USB, Ethernet, wireless such as WiFi/mesh network for results, and commands/controls. Comparing the differential water measurement data, computing the performance of the distillation system, determining whether distillation system is within specification, and displaying and/or monitoring the result and output may occur in real time with measuring the water in the water inflow line and the water outflow line by sensors and/or with receiving the inflow and outflow water measurement data.

The RWIDA processing 314 can be further controlled via the user interface processing 370 (by the user input and system controls of the user interface 260 including instruction for the inline differential water data analysis system or software update of the software for the processor) and/or the remote monitoring processing 360 (by the remote monitoring device 280 including instruction for the inline differential water data analysis system or software update of the software for the processor). The user input of commands and controls are fed back to the CPU to modify the computation and/or the operations. The control signals are used by the ND & CPU 320 and computation 330 to produce new results and data 340. These may all be done in real time.

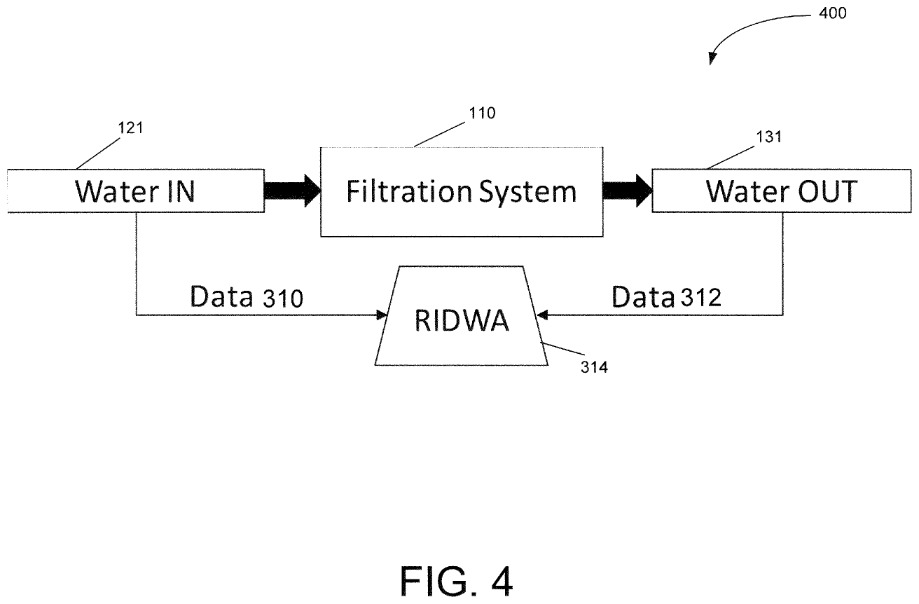

FIG. 4 is a diagram 400 illustrating differential water measurement data collection by the inline differential water data analysis according to an embodiment of the invention. A water stream flows from water IN 121 measured by the pre-sensor 120 through the filtration system 110 to water OUT 131 measured by the post-sensor 130. The pre-sensor 120 measures the water IN and provides inflow water measurement data (water IN data) 310 to the RIDWA 314. The post-sensor 130 measures the water OUT and provides outflow water measurement data (water OUT data) 312 to the RIDWA 314 for differential analysis. The inflow water measurement data 310 and outflow water measurement data 312 are obtained by measuring the water in the water inflow line 121 and the water outflow line 131 at about the same time, for example, within about a minute, or within a few seconds, or simultaneously. The pre-sensor 120 and post-sensor 130 are disposed inline along the water flow through the water IN line and the filtration system 110 and the water OUT without removing a water sample out of the water treatment system 110 or the water inflow line 121 or the water outflow line 131 for measurement or diverting the water flowing through the water inflow line 121 and the water treatment system 110 and the water outflow line 131 to a side stream for measurement.

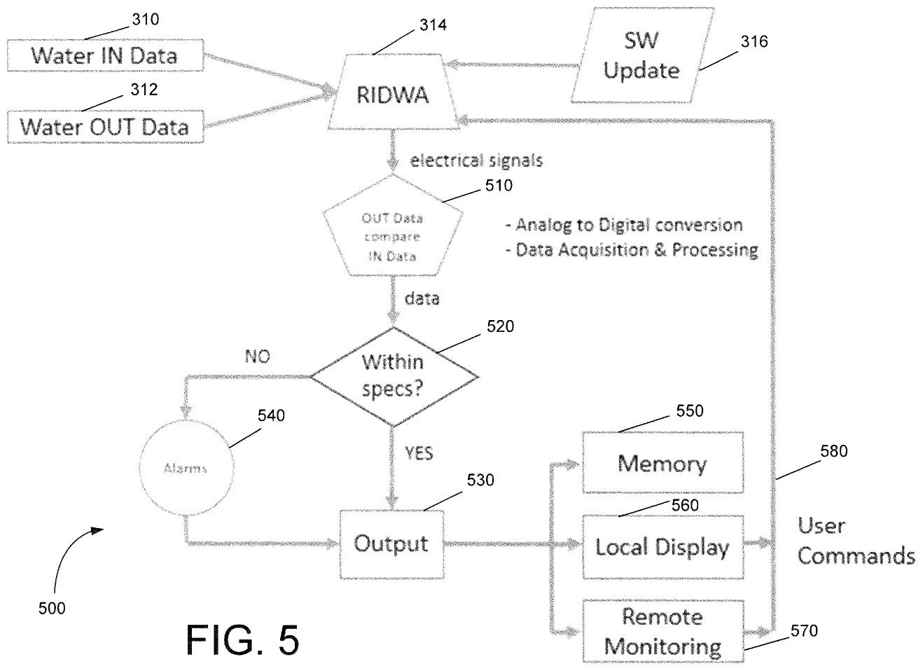

FIG. 5 is a system logic diagram 500 of the inline differential water data analysis system according to an embodiment of the invention. Water IN data 310 from the pre-sensor 120 and water OUT data 312 from the post-sensor 130 are inline measurement data that are received by the RIDWA 314, which also receives any software (SW) update 316.

The RIDWA 314 is programmed to process the input data via data analysis and evaluate the performance of the filtration system 110. In step 510, the RIDWA logic compares the inflow water measurement data 310 and the outflow water measurement data 312 to obtain a comparison, which involves analog-to-digital conversion and data acquisition and processing by A/D & CPU 320 of electrical signals, as performed by the data acquisition board 220 and I/O controller 230.

In step 520, the RIDWA logic computes a performance of the water filtration system 110 based on the comparison using a computation scheme and determines whether the comparison shows the performance of the filtration system 110 is within or outside specification(s), which involves specially programmed computation 330 by the microprocessor 232 using mathematics, business algorithm, operational logic, or the like. It involves computations of every single data point acquired including, for example, pH, conductivity, temperature, ORP, DO, etc. It also involves differential analyses of every data pair (same electrodes/probes) of IN and OUT sides. Performance analyses of the water filtration system 110 may produce results of rejection percentage (e.g., dissolved solids, salt, contaminants, etc.), filtration membrane health, pre-filters health, water pump health, etc.

Whether performance is within specification is determined by comparing it against the specification. The specification is in relation to the standard being measured against. For military, one may use TB MED 577 table 4.2 for short-term consumption. (https://armypubs.army.mil/epubs/DR_pubs/DR_a/pdf/web/tbmed577.pdf):

Such: 5<pH<9 AND TDS (conductivity)<1000 ppm AND turbidity<1 NTU.

Another example is the Environmental Protection Agency (EPA) for non-military requirements, secondary drinking water regulations and microbiology. (https://www.epa.gov/sites/production/files/2018-03/documents/dwtable2018.pdf):

Such: TDS (conductivity)<500 mg/L (ppm), 6.5<pH<8.5, turbidity<5 NTU.

In another example, North Atlantic Treaty Organization (NATO) uses STANAG 2136 (https://www.sdu.dk/-/media/files/om_sdu/institutter/iti/forskning/nato+arw/literature/amedp-4-.PDF):

Such: 5<pH<9.5 AND TDS (conductivity)<1500 ppm AND turbidity<1 NTU.

Having user-definable alerts allows for different standards and specifications to be used. Additionally, mission commanders may relax a particular specification based on risk assessment and mission needs, which would also necessitate a change to prevent the system from constantly alarming.

Differential measurement specification is set from common practice and the manufacturer. American Membrane Technology Association (AMTA) (respected RO/water treatment society) has rejection rates of 99.8% (https://www.amtaorg.com/). DuPont the manufacturer of the majority of the membranes used has rates of 95% typical up to +99% based on operating conditions. (https://www.dupont.com/content/dam/dupont/amer/us/en/water-solutions/public/documents/en/45-D01504-en.pdf).

In step 530, if the performance is within specification, the RIDWA logic generates output of the performance evaluation of the filtration system as conducted by the inline differential water data analysis and filtration system performance and diagnostics. If the performance is not within specification, one or more alarms are sounded in step 540 indicating existence of performance inadequacy of the water treatment system. The alarms may be audible (local) or visual (local or remote). In response, the RIDWA logic may be programmed to perform at least one of operation logic modification to modify operation logic of the inline differential water data analysis system, data computation modification to modify the computation scheme of the inline differential water data analysis system, or data logging modification to modify data logging of the inflow water measurement data and the outflow water measurement data by the inline differential water data analysis system. These modifications can be specially programmed already in the system software waiting to be activate/deactivate or can be made via software upgrade, when the performance of the water treatment system is outside the specification of the water treatment system.

The following are examples of operation logic modification:

1. Depending on other changing factors such as time of day, amount and speed of water and/or flow to be processed through the other system (water filtration), user/operator requests, and other parameters, the logic can modified to speed up or slow down the data sampling rates, data output rates, and hence data logging rates.

2. Depending on other factors such as water temperatures, turbidity, fluctuations of other measurements, water chemistry, membranes conditions, etc., the logic can be modified to change the order of parameters (pH, conductivity, ORP, DO, etc.) acquisitions (one before or after the other) to ensure accurate results.

3. Depending on operational situations such as security, localities, user/operator requests, system power fluctuations, data communication network changes, etc., the logic can be modified to disable/enable whole or part of path of data I/O and alarms. It can also be modified to change the encryption/decryption and languages of data output. It can be modified to log less or more of data, etc.

4. Depending on user requests, availability of other connected peripherals (different editions or versions of RIDWA) such as Global Positioning System (GPS), motion detection, cameras, microphone, speakers, different network options, etc., the logic can be modified to enable or disable more data of different types (locations, audio/visual, speech recognition for commands and control, different data I/O paths, etc.).

5. Depending on whether there are other RIDWAs on the same network, USB/Serial connection paths, etc., the logic can be modified to enable and disable group/cluster operations such as multiple data streams/alarms, remote monitoring sites, and data logging to multiple or unified site(s).

The following are examples of data computation modifications:

1. Depending on user/operator request, water conditions/chemistry, electrodes/probes manufacturer's specifications, parameter fluctuations (turbidity, pH, temperature, conductivity, etc.), the computation can be modified to adjust the mathematical calculations such as the electrodes (material) coefficients, using different/alternate numerical analyses to approximate the mathematical operations (exponential, logarithmic) for higher efficiency, better timing, greater accuracy, etc. (i.e., when some mathematical equations required large exponents that are too taxing for a low cost/low power CPU to compute in a timely manner, the computation can be modified to switch to different algebraic algorithms to approximate it, or some operations can be used to produce much lower/higher resolutions than the others and hence the need for better linear algebraic operations).

2. Depending on the results, the user request, the specifications, the parameters, the alarms, the amount/flow of water, etc., the computation can be modified to change the statistical methods or operations (averaging, mean, deviations, data scaling, sliding windows, etc.). The computation can also be modified to perform different statistical analyses to thin out or to capture better data happening moments.

3. Depending on particular A/D chipsets and/or different versions of the same/different CPUs being used (for particular versions of Arduino/Raspberry Pi computer boards), there are different hardware performance capabilities (8-bit/10-bit/12 bit, clock speed, RAM sizes, etc.) and the voltages/currents/resistance being acquired may yield lower resolutions, the computation mathematics can be modified to maintain a more consistent degree of accuracies.

4. Depending on the versions/editions of RIDWA with different attached peripherals, the computation can be modified to perform more data analyses (geospatial such as places, temporal such as time, critical alerts, relationships, etc.).

5. Depending on whether the RIDWA is alone or a part of a cluster of systems, the computation can be modified to enable/disable the computations for environmental operations (pollution, chemistry, etc.) such as analyses of a pond/lake/river/stream/etc. or a larger body of surface water.

The following are examples of data logging modifications:

1. Depending on the data sampling speed per above conditions, the data logging can be modified to change the amount of logged data to avoid over or under archiving of data (i.e., if there is a finite storage capacity, slower network bandwidth, etc.)

2. Depending on the security requirements, the data logging can be modified to enable/disable/strengthen the encryption level of logged data. It can disable remote data logging and store only locally.

3. Depending on the parameters, the user requests, operational conditions, etc., the data logging can be modified to perform/provide/output additional logging data information, such as statistical analyses data (average, mean, deviation, different data filters and scaling, etc.).

4. Depending on user requests, the data logging can be modified to send data (raw and/or analyzed) out to other networked remote sites for scientific/policy/environmental analyses by different partners/collaborators/communities.

5. Depending on planned additional system features and enhancements, the data logging can be modified to reformat the logged data output to support external mission command applications (geospatial and non-geospatial) such as engineering and intelligence to support military logistics as well as environmental protection in civil work.

In one example, conductivity is a sensor measurement processed by the differential water analyzer. The goal is to reduce conductivity through removal of dissolved ions. The threshold for 30-day military drinking water is:

Total TDS (ppm)<1000ppm

If the water remains above the limit, it can be collected and rerun through the water filtration system again, or it can be mixed with other water that is below the limit to achieve an average value that is below the stated limit. The latter is less desirable but produces more available water.

For reverse osmosis membrane health, an example of a specification is:

$$Total Rejection of conductivity or T D S \frac{( {ppm}_{in} - {ppm}_{out} )}{{ppm}_{i n}} > 98 \%$$

When the rejected water is not fully treated, the water can be collected and reprocessed; however, with a loss of water due to the rejection stream, which can also be collected and rerun, eventually each processing step will produce concentrated waste water that will be disposed of. This measurement provides an indication of equipment health. When a reverse osmosis membrane starts to fail, mechanically with holes or tears or use via pore opening, dissolved salts will pass through at a greater percentage. At a 98% rejection limit, normal sea water will be processed to 650-700 ppm which is under the required 1000 ppm for 30-day drinking limit. When this begins to reject less of the dissolved solids, the output will creep higher. It is possible for this to be within specification, but the water is still above the rejection limit. For example, if the water were from a sitting pool near the ocean, the TDS could be 55000 ppm due to evaporation; at 98% rejection, the produced water would be 1100 ppm, requiring a reprocessing step, but also indicating the system is performing as expected and is fully functioning.

In another example, ORP is a sensor measurement processed by the differential water analyzer. ORP is used to indicate whether there are materials in the water that are reducible via electrical exposure. Since reverse osmosis removes a portion of most materials (e.g., it is not perfect), ORP is suitable to check for removal of larger molecules. Measuring pre/post treatment provides assurance that the system is functioning and the water is safe to use after RO treatment. An example of a specification is:
If IN(mV) not zero, AND OUT(mV)=zero, THEN SAFE OR IF (IN(mV)=zero THEN SAFE ELSE
IF IN (mV) not zero, AND OUT(mV)not zero, THEN NOT SAFE
IF IN (mV)=zero, AND OUT(mV)not zero,THEN system needs attention

In another example, turbidity is a sensor measurement processed by the differential water analyzer. Turbidity is the amount of light scattered from debris that is suspended in water, such as dirt, microorganisms, and other particles. Measurement of turbidity is done before a RO membrane to ensure the water is not going to foul or clog the membrane. Measurement after RO treatment is taken to show compliance with water standards and that RO membranes are intact, installed correctly, and functioning. This is one measurement where it is possible to have more turbidity after treatment as a result of biological growth in a system. An example of a specification is:
If IN>ACTION LEVEL THEN alarm/alert
IF OUT>0.1 THEN THEN alarm/alert
IF IN<OUT, THEN system requires cleaning, sanitization, attention

The differential water analysis may evaluate the performance of the water filtration system based on multiple factors, such as conductivity, ORP, and turbidity. What is within specification will be based on a combination of the specifications of the multiple factors.

The output of the differential water analysis is stored in memory (storage 240) in step 550, shown on a local display for user interface (LCD user interface 260) in step 560, and/or transmitted for remote monitoring (remote monitoring device 280) in step 570. The RIDWA logic provides user commands via a feedback control line 580 from the user input and system controls of the user interface 260) and/or the remote monitoring device 280. The user commands are fed back to the CPU to modify the computation and/or the operations to produce new results and data. This feedback control may be done in real time.

The RIDWA 314 is portable and mobile and is characterized by system flexibility and versatility of the electro-mechanical design (hardware HW and software SW). By design, the RIDWA has a highest SWaP-C (Size, Weight and Power plus Cost) factor for a water quality monitoring system. It is very small, lightweight, low cost, and low power. It can be very easily attached to and used to test any drinking water production systems as a single-point displayed readout, localized, and in-situ sensor, and provide differential measurements data from two points in the process stream.

The RIDWA 314 is a complete, self-contained system with built-in microprocessors, A/D sensing for data acquisition, storage, and display. Thus, it does not need other interfacing computer and data processing systems, at the minimum. The result is a very mobile and portable device.

The RIDWA 314 has many connecting ways to access the water filtration data from the inside and to the outside systems. This would enable system survivability in case of partial failures. It uses Department of Defense (DoD)-approved data open-standards (non-proprietary) and is hence very flexible to access the data without proprietary software tools.

The RIDWA 314 is configured for serviceability. By design, the RIDWA electronics are very hardware-independent and thus can be easily serviced via COTS components as spare parts or enhancements (with the right SWaP-C factors) with software reprograming (on site or remotely). It can be repaired, customized, and adapted for the production systems to which it is attached.

The RIDWA 314 is extensible and expansible. Because the system is mostly software-defined with a very modern and advanced design, its functions and features can be extended and expanded. The RIDWA 314 can be upgraded with enhancements such as more data processing, more data analytics, and more data reporting capabilities. Also, the system can be modified to take advantage of better hardware that become available, such as faster chips, smaller probes, and more power efficient circuitries with just software changes.

The RIDWA 314 supports a broad range of user applications. The RIDWA system design makes adding additional peripherals simple such as GPS, different I/O Network (e.g., mesh or satellite), motion detectors, etc., to provide many additional capabilities complimenting the primary missions (water) such as security monitoring, multiple systems-integration communication, environmental mappings and monitoring, and the like.

In terms of system capabilities, the RIDWA 314 is configured to provide inline differential water data analysis based on water conductivity, salinity, dissolved ions/solids, and temperature measurements in single or differential modes. It is software upgradable for additional sensing of turbidity, pH, ORP, DO, chlorine, or other ions. It provides monitoring, performance validation, and diagnostics of DoD/COTS systems for water filtration, purification, and desalination. It provides real-time differential remote monitoring and data analyses. It is highly portable and compatible with other water filtration systems

In terms of operational environment, the RIDWA 314 can be used for surface or storage water testing. Potential customers include laboratories as well as industrial processes, such as Military Quarter Masters, Civil Works water-related programs, and public environmental programs.

The system hardware of the RIDWA 314 is characterized by low cost, low power, multiple data storage options, multiple I/O options, COTS probes compatibles, being network-enabled and reprogrammable, and additional sensing and features via software upgrade. The system software of the RIDWA 314 is characterized by data standards, real-time monitoring, modular extensibility, differential analyses, supporting geospatial and remote applications, software-driven calibrations, and software upgradability.

An embodiment of the computer-implementation of the inline differential water data analysis 140 has been shown in FIGS. 1-5. Different implementations are possible. Other embodiments may make use of different computer hardware and/or software configurations. A more general description of a computer device is described hereinbelow providing a variety of hardware and software elements that may be selected for implementing the inline differential water data analysis system.

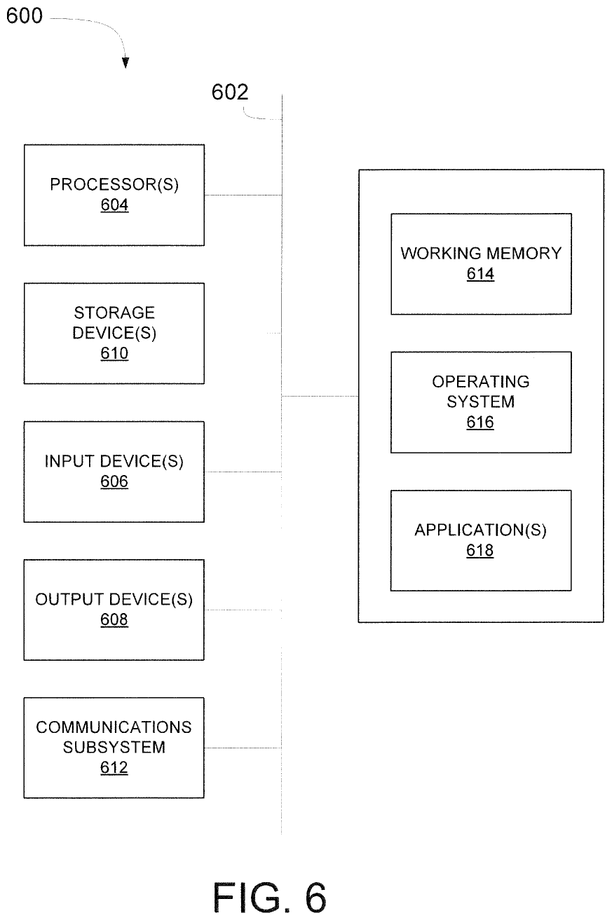

The computer device 600 of FIG. 6 is shown comprising hardware elements that may be electrically coupled via a bus 602 (or may otherwise be in communication, as appropriate). The hardware elements may include a processing unit with one or more processors 604, including without limitation one or more general-purpose processors and/or one or more special-purpose processors (such as digital signal processing chips, graphics acceleration processors, and/or the like); one or more input devices 606, which may include without limitation a remote control, a mouse, a keyboard, and/or the like; and one or more output devices 608, which may include without limitation a presentation device (e.g., controller screen), a printer, and/or the like. Input to the computer system 600 may be provided by analog-to-digital converters to convert the measurement signals from the pre-sensor 120 and post-sensor 130, and any other measurement devices into digital form for storage and/or processing. Separate external analog-to-digital devices can be attached to the bus 602 or communication subsystem 612 to provide measurements in digital form to the computer system 600 In some cases, an output device 608 may include, for example, a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 600 to a user. Output from the computer system 600 may be provided to digital-to-analog converters to send control signals from the computer to any motors or actuators. Digitally controlled motors or actuators may be attached to the bus 602 or communication subsystem 612 for digital control by the computer.

The computer system 600 may further include (and/or be in communication with) one or more non-transitory storage devices 610, which may comprise, without limitation, local and/or network accessible storage, and/or may include, without limitation, a disk drive, a drive array, an optical storage device, a solid-state storage device, such as a random access memory (RAM), and/or a read-only memory (ROM), which may be programmable, flash-updateable, and/or the like. Such storage devices may be configured to implement any appropriate data stores, including without limitation, various file systems, database structures, and/or the like.

The computer device 600 can also include a communications subsystem 612, which may include without limitation a modem, a network card (wireless and/or wired), an infrared communication device, a wireless communication device and/or a chipset such as a Bluetooth device, 802.11 device, Wi-Fi device, WiMAX device, cellular communication facilities such as GSM (Global System for Mobile Communications), W-CDMA (Wideband Code Division Multiple Access), LTE (Long Term Evolution), and the like. The communications subsystem 612 may permit data to be exchanged with a network (such as the network described below, to name one example), other computer systems, controllers, and/or any other devices described herein. In many embodiments, the computer system 600 can further comprise a working memory 614, which may include a random access memory and/or a read-only memory device, as described above.

The computer device 600 also can comprise software elements, shown as being currently located within the working memory 614, including an operating system 616, device drivers, executable libraries, and/or other code, such as one or more application programs 618, which may comprise computer programs provided by various embodiments, and/or may be designed to implement methods, and/or configure systems, provided by other embodiments, as described herein. By way of example, one or more procedures described with respect to the method(s) discussed above, and/or system components might be implemented as code and/or instructions executable by a computer (and/or a processor within a computer); in an aspect, then, such code and/or instructions may be used to configure and/or adapt a general purpose computer (or other device) to perform one or more operations in accordance with the described methods.

A set of these instructions and/or code can be stored on a non-transitory computer-readable storage medium, such as the storage device(s) 610 described above. In some cases, the storage medium might be incorporated within a computer system, such as computer system 600. In other embodiments, the storage medium might be separate from a computer system (e.g., a removable medium, such as flash memory), and/or provided in an installation package, such that the storage medium may be used to program, configure, and/or adapt a general purpose computer with the instructions/code stored thereon. These instructions might take the form of executable code, which is executable by the computer device 600 and/or might take the form of source and/or installable code, which, upon compilation and/or installation on the computer system 600 (e.g., using any of a variety of generally available compilers, installation programs, compression/decompression utilities, and the like), then takes the form of executable code.

It is apparent that substantial variations may be made in accordance with specific requirements. For example, customized hardware might also be used, and/or particular elements might be implemented in hardware, software (including portable software, such as applets, and the like), or both. Further, connection to other computing devices such as network input/output devices may be employed.

As mentioned above, in one aspect, some embodiments may employ a computer system (such as the computer device 600) to perform methods in accordance with various embodiments of the disclosure. According to a set of embodiments, some or all of the procedures of such methods are performed by the computer system 600 in response to processor 604 executing one or more sequences of one or more instructions (which might be incorporated into the operating system 616 and/or other code, such as an application program 618) contained in the working memory 614. Such instructions may be read into the working memory 614 from another computer-readable medium, such as one or more of the storage device(s) 610. Merely by way of example, execution of the sequences of instructions contained in the working memory 614 may cause the processor(s) 604 to perform one or more procedures of the methods described herein.

The terms “machine-readable medium” and “computer-readable medium,” as used herein, can refer to any non-transitory medium that participates in providing data that causes a machine to operate in a specific fashion. In an embodiment implemented using the computer device 600, various computer-readable media might be involved in providing instructions/code to processor(s) 604 for execution and/or might be used to store and/or carry such instructions/code. In many implementations, a computer-readable medium is a physical and/or tangible storage medium. Such a medium may take the form of a non-volatile media or volatile media. Non-volatile media may include, for example, optical and/or magnetic disks, such as the storage device(s) 610. Volatile media may include, without limitation, dynamic memory, such as the working memory 614.

Exemplary forms of physical and/or tangible computer-readable media may include a floppy disk, a flexible disk, hard disk, magnetic tape, or any other magnetic medium, a compact disc, any other optical medium, ROM, RAM, and the like, any other memory chip or cartridge, or any other medium from which a computer may read instructions and/or code. Various forms of computer-readable media may be involved in carrying one or more sequences of one or more instructions to the processor(s) 604 for execution. By way of example, the instructions may initially be carried on a magnetic disk and/or optical disc of a remote computer. A remote computer might load the instructions into its dynamic memory and send the instructions as signals over a transmission medium to be received and/or executed by the computer system 600.

The communications subsystem 612 (and/or components thereof) generally can receive signals, and the bus 602 then can carry the signals (and/or the data, instructions, and the like, carried by the signals) to the working memory 614, from which the processor(s) 604 retrieves and executes the instructions. The instructions received by the working memory 614 may optionally be stored on a non-transitory storage device 610 either before or after execution by the processor(s) 604.

It should further be understood that the components of computer device 600 can be distributed across a network. For example, some processing may be performed in one location using a first processor while other processing may be performed by another processor remote from the first processor. Other components of computer system 600 may be similarly distributed. As such, computer device 600 may be interpreted as a distributed computing system that performs processing in multiple locations. In some instances, computer system 600 may be interpreted as a single computing device, such as a distinct laptop, desktop computer, or the like, depending on the context.

A processor may be a hardware processor such as a central processing unit (CPU), a graphic processing unit (GPU), or a general-purpose processing unit. A processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512-bit, 256-bit, 128-bit, 64-bit, 32-bit, or 16-bit data operations.

Each of the calculations or operations discussed herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described herein. All features of the described systems are applicable to the described methods mutatis mutandis, and vice versa. Suitable tangible media may comprise a memory (including a volatile memory and/or a non-volatile memory), a storage media (such as a magnetic recording on a floppy disk, a hard disk, a tape, or the like; on an optical memory such as a compact disc (CD), a CD-R/W (Read/Write), a CD-ROM, a Digital Versatile Disc (DVD), or the like; or any other digital or analog storage media), or the like. While the exemplary embodiments have been described in some detail, by way of example and for clarity of understanding, those of skill in the art will recognize that a variety of modification, adaptations, and changes may be employed.

The present differential water analysis system or RIDWA system has applicability and usability in many areas. Examples of applications include diagnostics and evaluation of portable field water filtration systems when used in differential measurements mode, analyses of drinking water production, troubleshooting tools for water logistic engineers or military quarter masters, and water testing tools for long-term storages (water tanks, containers, etc.). Additional examples include military applications involving logistics and mission commands (battle/campaign planning), civil work involving municipalities and environmental monitoring and protection, and humanitarian assistance and disaster relief.

One example of environmental monitoring and protection involves environmental water monitoring in differential mode. It can be used to monitor the discharge vs. the input wastewater from city/county water treatment plants. It can be used to monitor the discharge vs. the surrounding surface water (lake, pond, stream, river, etc.). It can be used for city storm drain. Another example involves environmental water monitoring in dual-single mode. It can be used for spots or permanent water monitoring of natural surface water, industrial facilities, or agricultural sites. It can be used for water monitoring before and after and disasters (man-made or natural).

Environmental indicators contain information that may be chemically, mathematically, or biologically derived from the measured parameters, of chemically or biologically pre-cursors of the water quality information, or pollution, contamination, or toxicity information.

As basic RIDWA capabilities, water physical quality parameters may be (i) direct: pH, Electrical Conductivity (EC), Temperature, Dissolved Oxygen (DO); (ii) direct: Oxidation Reduction Potential (ORP); (iii) indirect: Total Dissolved Solids (TDS), Salinity, Acidity, Alkalinity; or (iv) indirect: Biochemical/Chemical Oxygen Demand (BOD/COD).

As upgraded RIDWA capabilities, water physical quality parameters may be (i) direct: Turbidity, Chlorine, Nitrate, Metal Ions (e.g., Manganese, Magnesium, Calcium, etc.); (ii) indirect: Color, Hardness, Nitrogen; or (iii) indirect: Toxic Inorganic/Organic (i.e., trihalomethanes THM, chloroform CHCl₃), Radioactive Elements, etc.

Examples of environmental implications include (i) pH (the acidity or alkalinity of water) with implications in aquatic life, corrosiveness of water, pollution indication; (ii) water temperature with implications in optimum levels for aquatic organisms and pollution indication; (iii) DO with implication in the right amount of oxygen which is essential to aquatic life but not too high/low, and pollution or contamination indication; (iv) turbidity (the amount of particulates suspended in water) with implications in transparency/clarity of water (e.g., cleanliness, essential for photosynthesis, for aquatic life) and pollution indication; (v) conductivity (ability of water to pass an electrical current) with implications in indication for inorganic dissolved solids, for waste water treatment, for salinity, and pollution indication (as in sewage leak, etc.); and (vi) nitrogen (NO3-N) with implications in what is essential for aquatic plants to grow and pollution (e.g., agriculture/industry run-off, contamination indication (excessive algae)).

As will be appreciated by one of ordinary skill in the art, the present invention may be embodied as an apparatus (including, for example, a system, a machine, a device, and/or the like), as a method (including, for example, a business process, and/or the like), as a computer-readable storage medium, or as any combination of the foregoing.

Embodiments of the invention can be manifest in the form of methods and apparatuses for practicing those methods.

Unless explicitly stated otherwise, each numerical value and range should be interpreted as being approximate as if the word “about” or “approximately” preceded the value or range.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

What is claimed is:

1. A differential water data analysis system comprising a data storage, a user interface, and a processor coupled to the data storage and programmed with software to:
receive inflow water measurement data from a water inflow line entering a water treatment system and outflow water measurement data from a water outflow line exiting the water treatment system, the inflow water measurement data and outflow water measurement data being obtained by measuring the water in the water inflow line and the water outflow line at about the same time and being received in real time;
compare the inflow water measurement data and the outflow water measurement data to obtain a comparison;
compute a performance of the water treatment system based on the comparison using a computation scheme;
determine whether the performance of the water treatment system is within or outside a specification of the water treatment system to produce a determination output; and
display the determination output in the user interface.

2. The differential water data analysis system of claim 1, further comprising:
a water inflow data line forming a releasable connection with a water inflow sensor disposed in the water inflow line to measure the inflow water measurement data; and
a water outflow data line forming a releasable connection with a water outflow sensor disposed in the water outflow line to measure the outflow water measurement data;
wherein the water inflow sensor and water outflow sensor are inline sensors to perform inline measurement of the water in the water inflow line and the water outflow line, respectively, the inflow water measurement data and outflow water measurement data being inline measurement data obtained without removing a water sample out of the water treatment system or the water inflow line or the water outflow line for measurement.

3. The differential water data analysis system of claim 1,
wherein the inflow water measurement data and the outflow water measurement data each comprise data relating to one or more water quality parameters; and
wherein the specification of the water treatment system comprises a mathematical expression which includes the one or more water quality parameters.

4. The differential water data analysis system of claim 1, wherein the processor is programmed, when the performance of the water treatment system is outside the specification of the water treatment system, to:
produce an alarm indicating existence of performance inadequacy of the water treatment system.

5. The differential water data analysis system of claim 1, wherein the processor is programmed, when the performance of the water treatment system is outside the specification of the water treatment system, to:
perform at least one of operation logic modification to modify operation logic of the differential water data analysis system, data computation modification to modify the computation scheme of the differential water data analysis system, or data logging modification to modify data logging of the inflow water measurement data and the outflow water measurement data by the differential water data analysis system.

6. The differential water data analysis system of claim 1, further comprising:
a data carrier configured to provide data communication with a remote monitoring device.

7. The differential water data analysis system of claim 6, wherein the processor is programmed to:
receive at least one of user input including instruction for the differential water data analysis system or software update of the software for the processor, from at least one of the user interface or the data carrier.

8. A portable differential water data analysis system comprising:
a data storage;
a user interface;
a water inflow data line forming releasable connection with a water inflow sensor disposed in a water inflow line entering a water treatment system to collect inflow water measurement data of the water treatment system;
a water outflow data line forming releasable connection with a water outflow sensor disposed in a water outflow line exiting the water treatment system to collect outflow water measurement data of the water treatment system, the inflow water measurement data and outflow water measurement data being obtained by measuring the water in the water inflow line and the water outflow line at about the same time and being collected in real time; and
a processor coupled to the data storage and programmed with software to
compare the inflow water measurement data and the outflow water measurement data to obtain a comparison;
compute a performance of the water treatment system based on the comparison using a computation scheme;
determine whether the performance of the water treatment system is within or outside a specification of the water treatment system to produce a determination output; and
display the determination output in the user interface.

9. The portable differential water data analysis system of claim 8,
wherein the inflow water measurement data and the outflow water measurement data each comprise data relating to one or more water quality parameters; and
wherein the specification of the water treatment system comprises a mathematical expression which includes the one or more water quality parameters.

10. The portable differential water data analysis system of claim 8, wherein the processor is programmed, when the performance of the water treatment system is outside the specification of the water treatment system, to carry out at least one of:
producing an alarm indicating existence of performance inadequacy of the water treatment system; or
performing at least one of operation logic modification to modify operation logic of the differential water data analysis system, data computation modification to modify the computation scheme of the differential water data analysis system, or data logging modification to modify data logging of the inflow water measurement data and the outflow water measurement data by the differential water data analysis system.

11. The portable differential water data analysis system of claim 8, further comprising:
a data carrier configured to provide data communication with a remote monitoring device.

12. The portable differential water data analysis system of claim 11, wherein the processor is programmed to:
receive at least one of (i) user input including instruction for the differential water data analysis system or (ii) software update of the software for the processor, from at least one of the user interface or the data carrier.

13. A differential water data analysis method for a differential water data analysis which includes a data storage, a user interface, and a processor programmed with software, the method comprising:
receiving inflow water measurement data from a water inflow line entering a water treatment system and outflow water measurement data from a water outflow line exiting the water treatment system, the inflow water measurement data and outflow water measurement data being obtained by measuring the water in the water inflow line and the water outflow line at about the same time and being received in real time;
comparing the inflow water measurement data and the outflow water measurement data to obtain a comparison;
computing a performance of the water treatment system based on the comparison using a computation scheme;
determining whether the performance of the water treatment system is within or outside a specification of the water treatment system to produce a determination output; and
displaying the determination output in the user interface.

14. The differential water data analysis method of claim 13,
wherein the inflow water measurement data and the outflow water measurement data are obtained by measuring the water in the water inflow line and the water outflow line simultaneously; and
wherein the receiving, comparing, computing, determining, and displaying occur in real time.

15. The differential water data analysis method of claim 13, further comprising:
forming a releasable communication with a water inflow sensor disposed in the water inflow line to measure the inflow water measurement data; and
forming a releasable communication with a water outflow sensor disposed in the water outflow line to measure the outflow water measurement data;
wherein the water inflow sensor and water outflow sensor are inline sensors to perform inline measurement of the water in the water inflow line and the water outflow line, respectively, the inflow water measurement data and outflow water measurement data being inline measurement data obtained without removing a water sample out of the water treatment system or the water inflow line or the water outflow line for measurement or diverting the water flowing through the water inflow line and the water treatment system and the water outflow line to a side stream for measurement.

16. The differential water data analysis method of claim 13,
wherein the inflow water measurement data and the outflow water measurement data each comprise data relating to one or more water quality parameters; and
wherein the specification of the water treatment system comprises a mathematical expression which includes the one or more water quality parameters.

17. The differential water data analysis method of claim 13, further comprising, when the performance of the water treatment system is outside the specification of the water treatment system:
producing an alarm indicating existence of performance inadequacy of the water treatment system.

18. The differential water data analysis method of claim 13, further comprising, when the performance of the water treatment system is outside the specification of the water treatment system:
performing at least one of operation logic modification to modify operation logic of the differential water data analysis system, data computation modification to modify the computation scheme of the differential water data analysis system, or data logging modification to modify data logging of the inflow water measurement data and the outflow water measurement data by the differential water data analysis system.

19. The differential water data analysis method of claim 13, further comprising:
providing data communication with a remote monitoring device.

20. The differential water data analysis method of claim 19, further comprising:
receiving at least one of user input including instruction for the differential water data analysis system or software update of the software for the processor, from at least one of the user interface or the data carrier.
